(19) United States
(12) Patent Application Publication
(10) Pub. No.: US 2019/0168017 A1
(43) Pub. Date: Jun. 6, 2019

(54) PHOTOTHERAPY DEVICE AND PHOTOTHERAPY METHOD

(71) Applicants: Public University Corporation Nagoya City University, Nagoya-city, Aichi (JP); Ushio Denki Kabushiki Kaisha, Tokyo (JP)

(72) Inventors: Akimichi MORITA, Nagoya-city, Aichi (JP); Hideyuki MASUDA, Tokyo (JP); Makoto KIMURA, Tokyo (JP)

(73) Assignees: Public University Corporation Nagoya City University, Nagoya-city, Aichi (JP); Ushio Denki Kabushiki Kaisha, Tokyo (JP)

(21) Appl. No.: 16/323,663

(22) PCT Filed: Feb. 6, 2017
(86) PCT No.: PCT/JP2017/004285

§ 371 Date: Feb. 6, 2019

(51) Int. Cl. A61N 5/06

(52) U.S. Cl. CPC A61N 5/0616; A61N 2005/0661; A61N 2005/0652

(57) ABSTRACT

Disclosed herein is a phototherapy device and a phototherapy method that employ an LED as a light source and are capable of achieving an appropriate therapeutic effect while suppressing a healthy portion of a skin from being damaged by light in a short wavelength band. The phototherapy device comprises a light source unit configured to irradiate an affected area with phototherapeutic light in a UV-B region. The light source unit includes an LED element configured to emit light in the UV-B region, and the LED element emits light having a peak wavelength equal to or greater than 312 nm. Also, the LED element preferably emits light having a peak wavelength equal to or less than 315 nm, and the LED element preferably emits light having a light emission spectrum of which full width at half maximum is equal to or less than 20 nm.

### TECHNICAL FIELD

The present invention relates to a phototherapy device and a phototherapy method that perform a therapy or treatment by use of light. More particularly, the present invention relates to a phototherapy device and a phototherapy method that medically treat a skin disease or a dermatosis or the like by ultra violet light in an UV-C (ultra violet C) region.

### BACKGROUND ART

Conventionally, ultra violet light is used to medically treat a skin disease or a dermatosis (such as a vitiligo vulgaris, a psoriasis, a palmoplantar pustulosis, an alopecia areata, an atopic dermatitis or the like). For example, a Patent Literature 1 (Japanese Patent Publication No. 4971665 B) discloses a certain phototherapy (or light treatment) device configured to treat a skin disease employing a light source consisting of an excimer lamp (more particularly, an excimer discharge lamp).

The excimer lamp encloses a predetermined luminous gas or the like inside a discharge vessel (arc tube). In particular, the excimer lamp is a lamp in which a pair of electrodes are disposed with at least one sheet of dielectric material (such as glass) intervening therebetween. The discharge via the dielectric material occurs by applying an alternating voltage to the pair of electrodes so as to cause the luminous gas to emit light.

The excimer lamp is configured to be able change a wavelength band (or wavelength range) of the emitted light (that is, ultra violet light) depending on a type of the enclosed gas. When xenon chloride (Xe—Cl) is enclosed therein as the discharge gas, it is possible to obtain ultra violet light having a peak in the vicinity of the wavelength of 308 nm.

The excimer lamp emits radiation light having a steep or sharp peak in the light emitting spectral characteristics thereof. For this reason, the excimer lamp has been considered to be advantageous as a light source for a therapeutic device that uses a specified wavelength only.

### LISTING OF REFERENCES

### Patent Literature

PATENT LITERATURE 1: Japanese Patent Publication No. 4971665 B

### SUMMARY OF THE INVENTION

### Problems to be Solved by the Invention

However, when lighting up the excimer lamp, it is required to apply a high voltage (20 kV to 80 kV) to the excimer lamp for its structural reason. In addition, in order to achieve a higher luminous efficiency, it is required to pulse light (or flash) the excimer lamp at the high frequencies. For this reason, as the technique disclosed in the above mentioned Patent Literature 1 (Japanese Patent Publication No. 4971665 B), when the excimer lamp is used as the light source for the phototherapy device, a larger space is required to be provided for accommodating a power supply unit thereof. As a result, it may entail a problem that an entire phototherapy device inevitably increases in size.

On the other hand, recent years, an LED (Light Emitting Diode) has been extensively developed. For this reason, the replacement of conventional lamps with the LEDs has been advanced not only for commonly used illuminations but also for the majority of manufacturing machining equipment or industrial machineries.

The higher output of the LEDs has become available and advanced not only in a visible light region but also in the ultra violet region. Even in the medical industry, the conventional light source has been expected to be replaced with the LEDs. In general, when the LED is to be used for the light source, it is expected to bring many benefits as more simplified circuit structure is achievable as compared to the conventional power supply unit for the lamp so that an entire device is expected to be downsized and light-weighted.

Under the above mentioned circumstances, in terms of the phototherapy device, it has been studied to employ the LED for the light source for the phototherapy device in place of the conventional excimer lamp. However, in reality so far the LED is considered not to be capable of being employed without further consideration because the safeness or safety applicable to medical devices has not yet sufficiently assured.

Unassured safeness or safety derives from the fact that the LED considerably differs from the conventional excimer lamp in the light emitting spectral characteristics of the emitted light. More particularly, while the conventional excimer lamp emits light having the full width at half maximum of its spectrum ranging between 3 nm to 5 nm, the LED emits light having the full width at half maximum of its spectrum largely ranging between 10 nm and 20 nm.

For this reason, assuming that the phototherapy device employs, as the light source, an LED of which peak wavelength is adjusted to coincide with the main peak wavelength of the conventional excimer lamp (308 nm) without particular consideration, such phototherapy device is assumed to emit a considerable amount of light in a short wavelength region that causes damage to a skin (hereinafter referred to as “light in a damaging wavelength region”). As a result, it is likely to cause a considerable amount of damages, which overwhelms the therapeutic effect thereof.

Taking the above mentioned circumstances into consideration, the present invention has been made in order to solve the above mentioned problems and an object thereof is to provide a phototherapy device and a phototherapy method that employ an LED element as a light source and are yet capable of suppressing a healthy portion of a skin from being damaged due to the light emitting spectral characteristics of the light source and achieving an appropriate therapeutic effect.

### Solution to Problems

In order to solve the above mentioned problems, according to one aspect of a phototherapy device of the present invention, there is provided a phototherapy device, comprising: a light source unit configured to irradiate an affected (or diseased) area with therapeutic light in a UV-B region; and an LED element provided at the light source unit and configured to emit light in the UV-B region, the LED element emitting light having a peak wavelength equal to or greater than 312 nm.

With the phototherapy device being so configured, by setting a lower limit of the peak wavelength of the light emitted from the LED element to be equal to or greater than 312 nm, it makes it possible to achieve an appropriate therapeutic effect while suppressing an adverse effect from being produced by the ultra violet light in the short wavelength region. Also, by employing the LED element as the light source, it makes it possible to allow an entire device to be downsized and light-weighted. In addition, by eliminating the high voltage being applied when lighting up the light source, it makes it possible to enhance the safeness the phototherapy device and to allow the phototherapy device to be used as a home-use device.

Furthermore, in the above described phototherapy device, the LED element may emit light having a peak wavelength equal to or greater than 313 nm. In this case, it makes it possible to suppress the adverse effect from being produced in more appropriate manner.

Yet furthermore, in the above described phototherapy device, the LED element may emit light having the peak wavelength equal to or less than 315 nm. In this case, it makes it possible to achieve a nearly equal (or similar) therapeutic effect to that of the conventional phototherapy device in a nearly equal treatment time to that of the conventional phototherapy device.

Yet furthermore, in the above described phototherapy device, the LED element may emit light having a light emitting spectrum of which full width at half maximum is equal to or less than 20 nm. In this case, it makes it possible to reduce light in the damaging wavelength region which is contained in the radiation light emitted from the LED element. As a result, it makes it possible to suppress the adverse effect from being produced by the ultra violet light in an appropriate manner.

Yet furthermore, in the above mentioned phototherapy device, the light source unit may directly radiate light emitted from the LED element as the therapeutic light without reducing the light emitted from the LED element. In this case, by eliminating a filter or the like to be provided, which reduces light emitted from the LED element, it makes it possible to prevent light in a desired (required) wavelength region for the therapy from being reduced due to cutting light in a specified wavelength region.

Yet furthermore, according to one aspect of a phototherapy method of the present invention, there is provided a phototherapy method of irradiating an affected area with therapeutic light in an UV-B region from a light source unit. The method comprising: a step of emitting light in a UV-B region having a peak wavelength equal to or greater than 312 nm from an LED element constituting a light source unit; and a step of irradiating an affected area with the light emitted from the LED element as therapeutic light. By doing this, it makes it possible to employ the LED element as the light source for the phototherapy and yet achieve an appropriate therapeutic effect while suppressing a healthy portion of a skin from being damaged by the light in a short wavelength region.

### Advantageous Effect of the Invention

According to a light therapy device and a light therapy method of the present invention, it makes it possible to suppress the healthy part of the skin from being damaged by the light in the short wavelength region so as to produce (achieve) the satisfactory therapeutic effect while employing the LED element as the light source for the light therapy.

The above mentioned and other not explicitly mentioned objects, aspects and advantages of the present invention will become apparent to a skilled person from the following embodiments (detailed description) when read and understood in conjunction with the appended claims and drawings.

### BRIEF DESCRIPTION OF DRAWINGS

FIG. 1 is a view schematically showing an exemplary configuration of a phototherapy device according to a present embodiment.

FIG. 2 is a view comparatively showing light emitting spectra of an excimer lamp and an LED.

FIG. 3 is a view showing a light emitting spectrum of a spectroscopic irradiator in which a peak wavelength is set to 310 nm.

FIG. 4 is a view showing measurement results indicative of a relationship between an irradiation dose and an apoptosis induction ratio.

FIG. 5 is a view showing an irradiation dose inducing 50% apoptosis.

FIG. 6 is a view showing measurement results of an adverse effect with the irradiation dose inducing 50% apoptosis.

FIG. 7 is a view showing a wavelength dependence of a therapeutic effect and an adverse effect.

FIG. 8 is a view showing comparative results of treatment time between a conventional device and the present embodiment.

### DESCRIPTION OF EMBODIMENTS

Hereinafter, embodiments of the present invention will be described with reference to the accompanying drawings in detail.

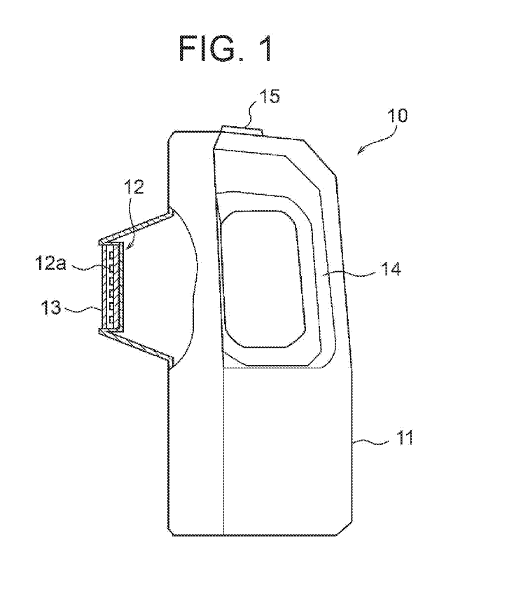

FIG. 1 is a view schematically showing a configuration of a phototherapy device 10 according to the present embodiment. The phototherapy device 10 according to the present embodiment is an LED skin medical treatment device that medically treats a skin disease by irradiating an affected are (or diseased area) with therapeutic light in a UV-B region (i.e., 280 nm to 320 nm) emitted from a light source unit including an LED element. The skin diseases to be medically treated may include a vitiligo vulgaris, a vitiligo psoriasis, a palmoplantar pustulosis, an alopecia areata, an atopic dermatitis, a parapsoriasis, a mycosis fungoides, a nodular prurigo, a malignant lymphoma and the like.

The phototherapy device 10 is provided with, as shown in FIG. 1 as a side view in which a partial cross-section thereof is shown, a housing 11, and a light source unit 12 arranged in the housing 11. The light source unit 12 is provided with LED elements (hereinafter also simply referred to as “LED”) that emit ultra violet light in a UV-B region.

Also, the phototherapy device 10 is provided with a light irradiance window 13 which allows light (radiation light) emitted from the LEDs 12a to transmit therethrough to be radiated as therapeutic light in front of the LEDs 12a (that is, at the left side in FIG. 1). The irradiance window 13 is constituted with a material made of a quartz glass or the like that allows the ultra violet light to transmit therethrough.

According to the present embodiment, the light source unit 12 may have a structure in which a plurality of LEDs 12a are arranged in an array. The number of the LEDs 12a can be determined as appropriate depending on a size of an irradiation area of the therapeutic light radiated from the phototherapy device 10. Also, each of the plurality of LEDs 12a may be capable of individually controlling an optical output thereof. For example, in the irradiation area of the therapeutic light, an individual optical output of each LED 12a may be controlled such that an illuminance (light intensity) on an irradiation area is adjusted to be uniform. Yet also, only a part of the plurality of LEDs 12a may be turned on so as to allow the size or a shape of the irradiation area of the therapeutic light to correspond to the size and a shape of the affected area.

Yet furthermore, the phototherapy device 10 is provided with a gripper (or handle) 14 at an opposite side to the irradiance window 13. In addition, the phototherapy device 10 is provided with a switch 15 at an upper portion of the gripper 14. An operator, who operates the phototherapy device 10 (for example, a doctor), holds the phototherapy device 10 by grasping the gripper 14, and lights up (turns on) the LEDs 12a by pushing down the switch 15 in a state in which the irradiance window 13 at a front face abuts onto the affected area of a patient or is proximately positioned thereto so as to irradiate the affected area with the therapeutic light.

LEDs 12a have the peak wavelength within a range equal to or greater than 312 nm and equal to or less than 320 nm, preferably within a range equal to or greater than 313 nm and equal to or less than 320 nm. Also, more preferably, the above described peak wavelength has an upper limit equal to 315 nm. Yet also, the full width at half maximum (half-width) of the light emitting spectrum thereof is equal to or less than 20 nm.

By employing the LEDs 12a having the peak wavelength within the above described range, it makes it possible to achieve (or produce) an appropriate therapeutic effect while suppressing a healthy portion of a skin from being damaged. Hereinafter, this action mechanism will be more particularly described.

In the conventional phototherapy device employing the conventional excimer lamp, ultra violet light having a peak at the wavelength of 308 nm is used in order to produce a sufficient therapeutic effect in a condition in which an adverse effect (or side effect) is suppressed not to be more than the certain extent. Thus, when trying to replace the excimer lamp with the LED for the light source for the phototherapy device, it may be first conceivable to simply employ an LED light having a peak at the wavelength of 308 nm as well.

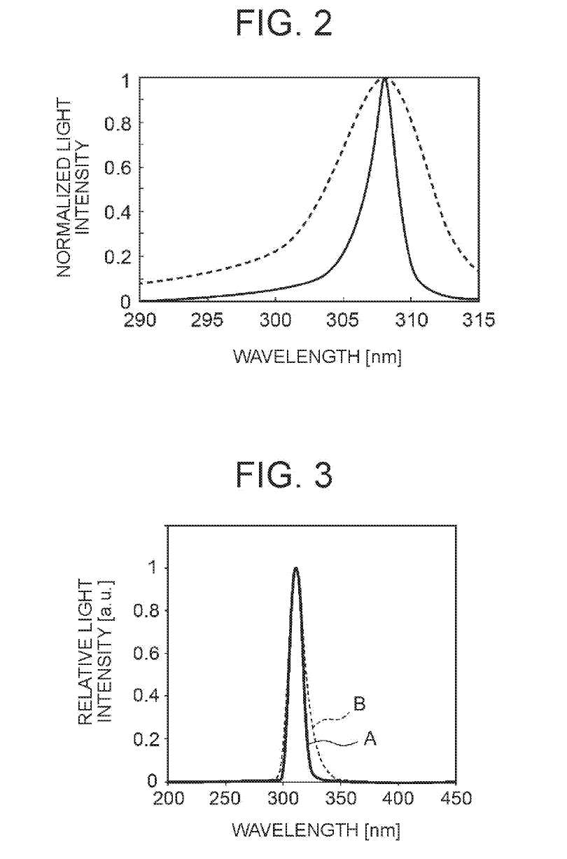

However, when the light emitting spectra are compared to each other between the excimer lamp and the LED with both peak wavelengths coinciding with each other, as shown in FIG. 2, both shapes of both light emitting spectra considerably differ from each other. More particularly, as shown in the solid line in FIG. 2, the excimer lamp has a sharp peak and a little light radiation is observed at the skirts (that is, the plains at the feet) of the light emitting spectrum. On the other hand, as shown in the dashed line in FIG. 2, the LED emits light having a broad light emitting spectrum and the emitted light contains more light in the damaging wavelength region having the wavelength equal to or less than 297 nm. More particularly, when comparing both full widths at half maximum of both light emitting spectra, while the light emitted from the excimer lamp has the full width at half maximum ranging between 3 nm and 5 nm, the light emitted from the LED has the full width at half maximum ranging between 10 nm and 20 nm.

In other words, when employing the LED of which peak wavelength is set to coincide with the main peak wavelength of the conventional excimer lamp (308 nm), the skirt of the light emitting spectrum thereof inevitably extends to the damaging wavelength region. For this reason, it is anticipated that the damage (in other words, adverse effect) overwhelms (exceeds) the therapeutic effect, if the light source for the phototherapy device is simply changed from the conventional excimer lamp to the LED of which peak wavelength is set to coincide with the main peak wavelength (308 nm) of the excimer lamp without further consideration. Same applies to the case in which the peak wavelength of the LED is set to coincide with the main peak wavelength of the conventional fluorescent lamp (311 nm).

According to the present embodiment, the peak wavelength of the LED is not set to coincide with the main peak wavelength of the conventional excimer lamp (308 nm). Instead, a lower limit of the peak wavelength of the LED light is set to 312 nm, preferably to 313 nm. As a result, it makes it possible to achieve a sufficient therapeutic effect while suppressing the adverse effect from being produced. In addition, by employing an LED having the full width at half maximum of the light emitting spectrum equal to or less than 20 nm, it makes it possible to reduce the light in the damaging (harmful) wavelength region which is contained in the emitted light. As a result, it makes it possible to suppress the adverse effect due to the ultra violet light from being produced in more appropriate manner. Hereinafter, experimental examples will be described in connection with the above described content.

The inventors of the present invention conducted experiments below using a phototherapy device employing a light source having the following light emitting spectra in order to investigate the wavelength dependence of the therapeutic effect and the wavelength dependence of the adverse effect in a phototherapy device employing the LED light source.

### <Conditions>

Full Width at Half Maximum of Light Emitting Spectrum: 14 nm

Peak Wavelength: 280 nm, 285 nm, 290 nm, 295 nm, 300 nm, 305 nm, 310 nm, 315 nm, 320 nm (in increments of 5 nm in the UV-B region)

As the light source of the experimental device, a spectroscope (spectroscopic) irradiator was used that is capable of forming light similar to the light emitted from the LED and arbitrarily (and continuously) setting the peak wavelength.

FIG. 3 shows the light emitting spectra of the spectroscope irradiator in which the full width at half maximum was set to 14 nm and the peak wavelength was set to 310 nm. In FIG. 3, a curved line A denotes a light emitting spectrum of the spectroscope irradiator and a curved line B denotes a light emitting spectrum of a representative LED in which the full width at half maximum was set to 14 nm and the peak wavelength was set to 310 nm.

### (Experiment 1)

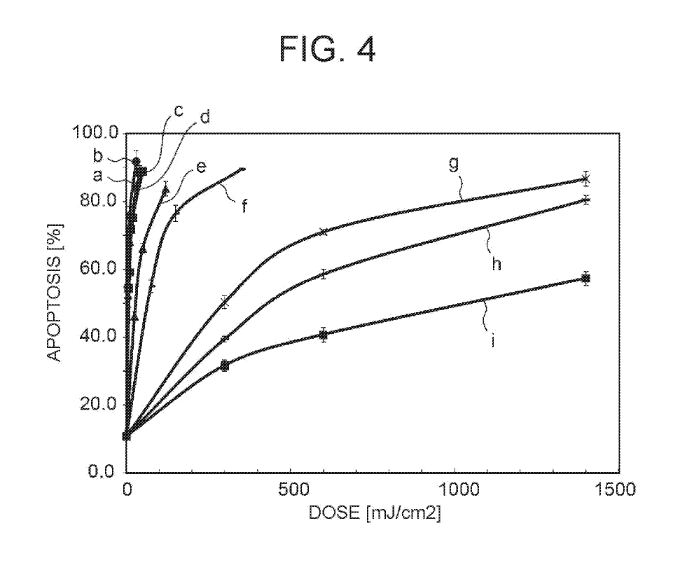

Jurkat cells having a concentration of 4*10⁵/mL were disseminated in 24-well plate in a unit of 500 μL to each well, and the spectroscope irradiator was used to irradiate the Jurkat cells with rays of light under the above described conditions, respectively. Subsequently, FACS (Fluorescence Activated Cell Sorting) was used to measure a ratio of the Annexin V positives, in other words, the apoptosis induction ratio. FIG. 4 shows the experimental results thereof.

In FIG. 4, the horizontal axis denotes the light irradiation dose (mJ/cm²) and the vertical axis denotes the apoptosis induction ratio. In FIG. 4, a curved line “a” denotes a relationship between the light irradiation dose of the light having the peak wavelength of 280 nm and the apoptosis induction ratio. Similarly, Relationships are shown in FIG. 4 between the apoptosis induction ratios and the light irradiation doses of the rays of light having the peak wavelength of, with a curved line b, 285 nm, with a curved line c, 290 nm, with a curved line d, 295 nm, with a curved line e, 300 nm, with a curved line f, 305 nm, with a curved line g, 310 nm, with a curved line h, 315 nm, and with a curved line i, 320 nm, respectively.

As apparent from FIG. 4 as well, it is observed that pseudo LED light at the short wavelength side, such as the light having the peak wavelength of 280 to 300 nm (that is, curved lines “a” to “e”), yields a higher apoptosis induction ratio with a smaller light irradiation dose. In other words, a higher therapeutic effect is achievable with a smaller light irradiation dose.

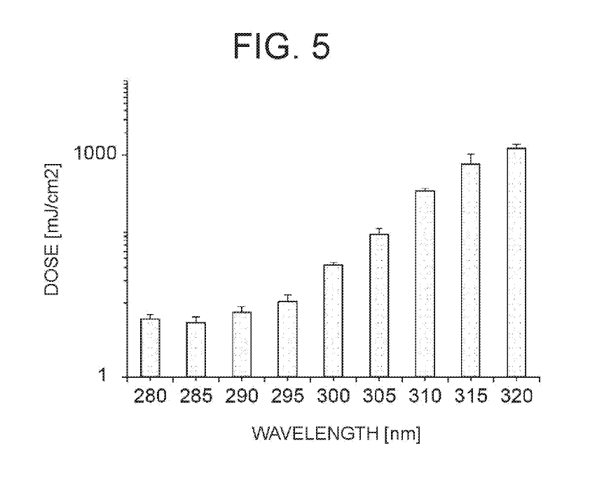

In order to further compare the therapeutic effects by the rays of light under the above described conditions, line irradiation doses were measured which were required to achieve similar (equivalent) therapeutic effects by the rays of light, respectively. More particularly, each experiment for measuring the above described apoptosis induction ratio was performed multiple times (for example, three times), and an irradiation dose that yielded the apoptosis induction ratio of 50% was measured. FIG. 5 shows the experimental result thereof. In FIG. 5, the horizontal axis denotes the peak wavelength of the pseudo LED light radiated, and the vertical axis denotes the light irradiation dose that induces the apoptosis of 50%. In FIG. 5, an average value of the irradiation doses measured multiple times is tagged with an error bar of the standard error.

As described above, when simply considering the therapeutic effect only, it is observed that the LED light at the above described short wavelength side is more effective. On the other hand, however, such LED light at the above described short wavelength side is also observed to produce a larger adverse effect due to the ultra violet light radiation. For this reason, it is impossible to be used as the therapeutic light.

In light of the above described observations, the inventors of the present invention further conducted the following experiments assuming that appropriate light as the therapeutic light is light that produces the smallest adverse effect with the equivalent (similar) therapeutic effect being obtainable out of the rays of light under the above described conditions.

### (Experiment 2)

Jurkat cells having a concentration of 4*10⁵/mL were disseminated in 24-well plate in a unit of 500 μL to each well, and the spectroscope irradiator was used to irradiate the Jurkat cells with rays of light under the above described conditions, respectively. The light irradiation dose was set to the light irradiation dose that induces the apoptosis of 50%, which was obtained through the Experiment 1. The following Table 1 shows light irradiation doses of the respective rays of light. It should be noted that the light irradiation dose shown in the Table 1 shows an average value of the irradiation doses that produce the apoptosis induction ratio of 50% which were measured multiple times for each of the rays of light.

TABLE 1

| Peak Wavelength [nm] | 50% apoptosis light irradiation dose [mJ/cm²] |
|---|---|
| 280 | 6 |
| 285 | 5 |
| 290 | 7 |
| 295 | 10 |
| 300 | 32 |
| 305 | 84 |
| 310 | 326 |
| 315 | 752 |
| 320 | 1232 |

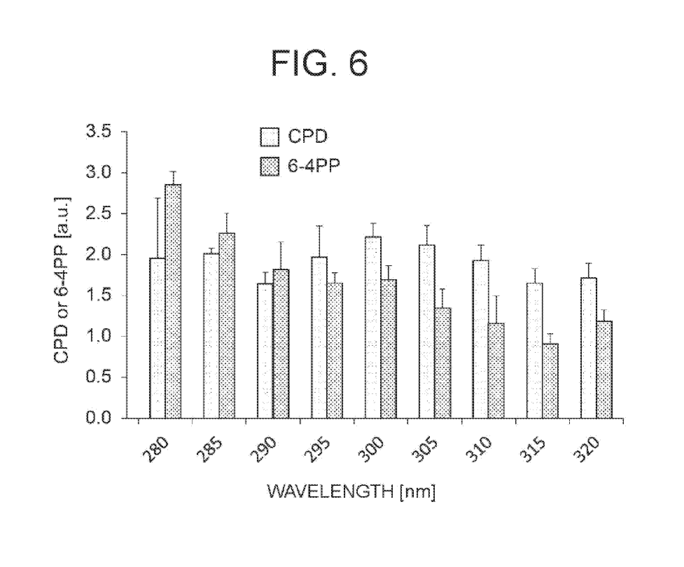

Subsequently, right after being irradiated, the cells were immediately recovered and DNAs were extracted from the cells, and then the production quantity of the CPD (cyclobutane type pyrimidine dimer) and 6-4PP (6-4 type photoproduct) were measured by the ELISA (Enzyme-Linked Immuno Sorbent Assay) method. FIG. 6 shows the experimental results thereof.

As apparent from FIG. 6, it was observed that an amount (quantity) of DNA damages induced by the ultra violet light becomes the smallest when irradiated with the light having the peak wavelength of 315 nm. In light of this finding, further verifications were repetitively performed seeking for the permissible (allowable) wavelength range in the wavelength region in the vicinity of the peak wavelength of 315 nm.

First, in the relationship diagram between the light irradiation dose and the apoptosis of the respective rays of light under the above described conditions (FIG. 4), a slope in each of the linear regions was defined as an action coefficient. Similarly, relationship diagrams between the light irradiation doses and the CPD of the rays of light were obtained for the rays of light, respectively, and a slope in each of the linear regions was defined as an action coefficient. Then, those action coefficients were normalized (standardized), respectively, such that respective maximum values were normalized to be 1.

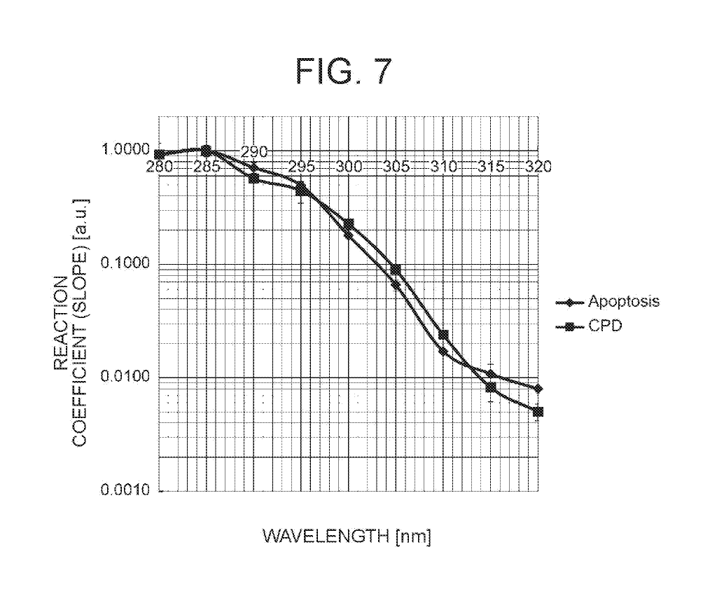

FIG. 7 is a view plotting the action coefficients of the apoptosis and the action coefficients of the CPD, respectively. A curved line obtained by plotting the action coefficients of the apoptosis is indicative of an action curve of the therapeutic effect. On the other hand, a curved line obtained by plotting the action coefficients of the CPD is indicative of an action curve of the adverse effect. As apparent from FIG. 7, it is observed that a particular wavelength range that allows the therapeutic effect to overwhelm (exceed) the adverse effect is a range of the peak wavelength ranging between 285 nm and 297 nm, and also a range of the peak wavelength equal to or greater than 312 nm.

Amongst those wavelength ranges in which the therapeutic effect overwhelms the adverse effect, the range of the peak wavelength ranging between 285 to 297 nm has, as shown in FIG. 6, a larger absolute value of the adverse effect due to the ultra violet light. In other words, it is assumed that the light in the range of the peak wavelength ranging between 285 and 297 nm has a relatively smaller therapeutic effect as compared to the magnitude of the adverse effect. As a result, it is assumed that the light having the peak wavelength equal to or greater than 312 nm is more effective for the therapeutic light that is capable of achieving a sufficient therapeutic effect while suppressing the adverse effect from being produced.

It should be noted that, in FIG. 7, the peak wavelength at which two action curves intersect with each other at the long wavelength side lies between 312 nm and 313 nm in a precise sense. Nevertheless, as FIG. 7 was obtained by plotting median values including errors, it can be assumed that the lower limit of the permissible wavelength range is the peak wavelength of 312 nm. However, in order to suppress the adverse effect from affecting more efficiently, it is preferable to set the lower limit of the peak wavelength to 313 nm.

In addition, the peak wavelength of the light that is effective as the therapeutic light is preferably equal to or less than 315 nm. As shown in FIG. 5, the light having the peak wavelength exceeding 315 nm requires a larger 50% apoptosis irradiation dose. For this reason, when employing the light within this range as the therapeutic light, it makes time required for the treatment longer inevitably. In this regard, in light of the circumstance at a common clinical site, even when the LED is employed as the light source, it is desirable to limit the treatment time up to similar time to the treatment time when the narrow band UVB (NB-UVB) treatment method is used, or at most double thereof. Here the NB-UVB treatment method is referred to as a treatment method that irradiates an affected area only with the ultra violet light in the medium wavelength ultra violet (UV-B) region. For the ultra violet light source in the NB-UVB treatment method, it is common to employ a lamp having a light emitting spectrum in the narrow band region ranging between 311 and 313 nm.

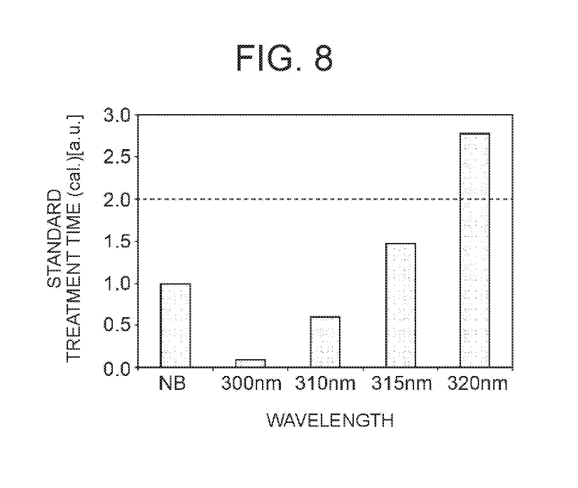

FIG. 8 shows comparative results obtained by estimating the treatment time when treating by use of the LED light having the respective peak wavelengths (300 nm, 310 nm, 315 nm, and 320 nm), and comparing the estimated treatment time to the treatment time when treating by use of the NB-UVB (“NB” in FIG. 8). Here, it is assumed that irradiances of both light sources are the same with each other. As shown in FIG. 8, the treatment time when employing the LED light having the peak wavelength of 320 nm reaches to two and a half times or more of the treatment time when employing the narrow band UVB.

On the other hand, the treatment time when employing the LED light having the peak wavelength of 315 nm does not exceed double of the treatment time when employing the narrow band UVB. For this reason, considering the duration of the treatment time, it is preferable to set the peak wavelength of the light effective for the therapeutic light to be equal to or less than 315 nm.

As described above, reactions (responses) of the cells irradiated with the ultra violet light were verified through the above described experiments. As a result, when the LED is used as the light source, it was confirmed that, as long as the LED light has the peak wavelength range equal to or greater than 312 nm and equal to or less than 315 nm, it makes it possible to achieve a sufficient therapeutic effect while suppressing the adverse effect from being produced, even when such LED light is directly used as the therapeutic light without cutting (filtering) light having a specified wavelength by a filter or the like.

Considering the above described experimental results, according to the present embodiment, the phototherapy device 10 sets the lower limit of the peak wavelength of the LED light, which is to be used for the therapeutic light, to be 312 nm. With the device being so configured, it makes it possible to achieve a sufficient therapeutic effect while suppressing the adverse effect from being produced due to the ultra violet light.

It should be noted however, as a separate thought from the above described one, it might be conceivable to use a filter to cut light at a skirt (that is, a plain at a foot) of a light emitting spectrum that reaches to the damaging wavelength region, while employing the LED of which peak wavelength is set to coincide with a main peak wavelength of the conventional excimer lamp (308 nm). However, in this case, the following problems may be entailed.

Methods of selecting the light wavelength by a filter (in other words, light shielding methods) fall into two types. One type is to use a colored glass, and the other type is to use an interference film (multilayer film).

In case of the filer using the colored glass, it is considered to be impossible to design the filter such that the filter sharply eliminates light at a specified wavelength band. Thus, the filter is necessarily designed so as to broadly cut light. For this reason, in order to cut light at the damaging wavelength band, it has to sacrifice light at the desired wavelength band for the treatment to be inevitably cut as well. When the light at the desired wavelength band for the treatment is reduced by the filter, the irradiance is also reduced so as to make the treatment time longer. Although the efficiency of the LED becomes higher and higher, the LED is not yet attain a high light output as compared to the excimer lamp or the mercury lamp. Thus, it is problematic if the light at the desired wavelength band for the treatment is reduced.

On the other hand, in case of the filer using the multilayer film, it is considered to be possible to design the filter such that the filter sharply selects light at the desired wavelength band only to be radiated. However, due to the characteristics of the multilayer film itself, the wavelength of the light transmissive therethrough inevitably varies depending on an incidence angle of the light with respect to the film. When employing the LED, it is difficult to control the incidence angle of the LED light with respect to the multilayer film. For this reason, it is impossible to cut the wavelength as originally designed.

As described above, cutting the light in the damaging wavelength region only without cutting the light at the desired wavelength band for the treatment. As a result, when employing the LED of which peak wavelength is set to coincide with the main peak wavelength of the conventional excimer lamp (308 nm), it is hardly attainable to achieve the sufficient therapeutic effect while suppressing the adverse effect from being produced.

On the other hand, according to the present embodiment, as the LED, for the light source, of which peak wavelength has the lower limit set to 312 nm (preferably 313 nm) is employed, as apparent from the above described experimental results, it makes it possible to achieve the sufficient therapeutic effect while suppressing the adverse effect from being produced even without using the filter. In other words, even when the light emitted from the LED is directly used as the therapeutic light without reducing the light at the desired wavelength band for the treatment, it makes it possible to achieve the sufficient therapeutic effect while suppressing the adverse effect from being produced.

Furthermore, the phototherapy device 10 according to the present embodiment sets the upper limit of the peak wavelength of the LED light, which is used as the therapeutic light, to 315 nm. Thus, it makes it possible to achieve the nearly equal (similar) therapeutic effect to that of the NB-UVB treatment method with nearly equal treatment time to that of the NB-UVB treatment method. It should be noted that the upper limit of the peak wavelength of the LED light is permissible to be up to 320 nm. In this case, although the longer treatment time is required as compared to the NO-UVB treatment method, still it makes it possible to achieve the nearly equal therapeutic effect to that of the NV-UVB treatment method.

As described above, the phototherapy device 10 according to the present embodiment directly induces the pathogenetic immune cells (T cells) to the apoptosis by irradiating the affected area affected by the skin disease with the therapeutic light and sedates (calms down) the overreacting affected area so as to achieve the sufficient therapeutic effect. In particular, the phototherapy device 10 according to the present embodiments is capable of, while employing the LED element as the light source, suppressing the healthy portion of the skin from being damaged by the light in the short wavelength region and also achieving the sufficient therapeutic effect.

### Modification to Embodiments

In the above described embodiments, a certain case has been described in which the light emitted from the LED is directly radiated without being intervened by the filter which reduces the emitted light at a specified wavelength. However, alternatively, in order to further increase the safety, the filter may be used to cut the light in the damaging wavelength region contained in the light emitted from the LED. In this case, as described above, although the longer treatment time is required as the light at the desired wavelength band for the treatment is also reduced, it makes it possible to achieve the sufficient therapeutic effect while suppressing the adverse effect from being produced in an efficient manner.

Although specific embodiments are described above, these embodiments are merely illustrative in nature and are not intended to limit the scope of the present invention. The apparatuses and the methods described in the present specification can be implemented in embodiments aside from those described above. Omissions, substitutions, and modifications can be made, as appropriate, to the embodiments described above without departing from the scope of the present invention. An embodiment with such omissions, substitutions, and modifications is encompassed by what is described in the claims and any equivalent thereof and falls within the technical scope of the present invention.

### REFERENCE SIGNS LIST

10: Phototherapy Device
11: Housing
12: Light Source Unit
12a: LED Element
13: Light Irradiation Window
14: Gripper
15: Switch

1. A phototherapy device, comprising:
a light source unit configured to irradiate an affected area affected by a skin disease with phototherapeutic light in a UV-B (ultra violet B) region; and
an LED element provided at the light source unit and configured to emit light in the UV-B region,
the LED element emitting light having a peak wavelength equal to or greater than 312 nm.

2. The phototherapy device according to claim 1, wherein, the LED element emits light having a peak wavelength equal to or greater than 313 nm.

3. The phototherapy device according to claim 1, wherein, the LED element emits light having a peak wavelength equal to or less than 315 nm.

4. The phototherapy device according to claim 1, wherein, the LED element emits light having a light emission spectrum of which full width at half maximum is equal to or greater than 10 nm and equal to or less than 20 nm.

5. The phototherapy device according to claim 1, wherein, the light source unit directly radiates the light emitted from the LED element as the phototherapeutic light without reducing the light emitted from the LED element.

6. The phototherapy device according to claim 1, wherein, the light source unit has a structure in which a plurality of LED elements are arranged in an array, and each of the plurality of LED elements emits light having the peak wavelength equal to or greater than 312 nm.

7. A phototherapy method of irradiating an affected area affected by a skin disease with phototherapeutic light in a UV-B region emitted from a light source unit, comprising:
a step of emitting light in the UV-B region having a peak wavelength equal to or greater than 312 nm from an LED element constituting the light source unit; and
a step of irradiating the affected area with the light emitted from the LED element as the phototherapeutic light.
